(19) United States
(12) Patent Application Publication
(10) Pub. No.: US 2018/0078659 A1
(43) Pub. Date: Mar. 22, 2018

(54) METHODS OF DETECTING BIOLOGICAL ACTIVITY, CELLULAR BEHAVIOR AND DRUG DELIVERY USING ENCAPSULATED POLYMETHINE AGGREGATES

(71) Applicants: Nanohybrids, Austin, TX (US); Board of Regens, The University of Texas System, Austin, TX (US)

(72) Inventors: Stanislav Emelianov, Austin, TX (US); Konstantin Sokolov, Missouri City, TX (US); Geoff Luke, Austin, TX (US); Carolyn Bayer, Austin, TX (US); Justin Harris, Austin, TX (US); Albert Lee, Houston, TX (US)

(21) Appl. No.: 15/666,750

(22) PCT Filed: Feb. 7, 2016
(86) PCT No.: PCT/US2016/016916

§ 371 Date: Aug. 2, 2017

Related U.S. Application Data
(60) Provisional application No. 62/113,477, filed on Feb. 8, 2015.

(51) Int. Cl. A61K 49/00; A61B 5/00; G01N 21/64; G01N 33/58

(52) U.S. Cl. CPC A61K 49/0034; A61B 5/0095; A61B 5/0071; A61B 5/4848; A61B 5/0084; G01N 2021/6439; A61K 49/0021; A61K 49/0084; G01N 21/6428; G01N 33/582; A61K 49/0032

(57) ABSTRACT

Presented herein are methods of using encapsulated J-aggregates of indocyanine green (ICG) as a ratiometric sensor of biological activity. Upon interaction with a biological phenomenon of interest, the encapsulated J-aggregates can be released and dissolved upon rupture, inducing a detectable hypsochromic shift in the absorption spectra and corresponding increase in fluorescence. Various imaging techniques can be employed to visualize this sensor including photoacoustic imaging, two-photon imaging, fluorescence imaging, near infrared imaging, and a variety of other optical or optics-based techniques. Additionally, if the J-aggregates of ICG are also encapsulated with drugs or therapeutic molecules, the ratiometric sensing using ICG can be used to confirm drug release and delivery.

### CROSS-REFERENCE DATA

This patent application is a national phase filing of the PCT patent application No. PCT/US2016/016916 filed 7 Feb. 2016, which in turn claims priority date benefit from a U.S. Provisional Patent Application No. 62/113,477 filed 8 Feb. 2015 with the same title and incorporated herewith in its entirety by reference.

### GOVERNMENT LICENSE RIGHTS STATEMENT

This invention was made with government support under SBIR contract grant No. HHSN268201400039C entitled “Molecularly Targeted Liposomes for Detection of Macrophages in High Risk Artherosclerotic Plaque” and awarded by the National Heart, Lung and Blood Institute of The National Institutes of Health. The government has certain rights in the invention.

### BACKGROUND

Theranostic therapies—treatments that can diagnose and deliver targeted therapy—are invaluable instruments in the fight against diseases, such as cancer and atherosclerosis. Nanoparticles can be loaded with drugs and contrast agents, targeted to affect specific proteins, and monitored via molecular imaging for cellular uptake and drug release. This targeted technique has less detrimental side effects than general treatments due to the preservation of healthy cells.

Molecular imaging requires development of specific contrast-enhancing agents that can provide information about the distribution and activity of the cells and biomolecules involved at various stages of disease progression. The field of nanomaterials has introduced a variety of promising contrast-enhancing nanoparticles across different disciplines and imaging modalities. These new nanomaterials offer increased signal intensity, improved imaging contrast and superior binding affinity in comparison to “traditional” contrast agents (e.g. molecular-based contrast agents and monomeric dyes). Likewise, nanotechnology offers unique benefits due to its ability to interact with biological processes at the cellular and molecular level.

Liposomal nanoparticles are of interest due to their biocompatible, nontoxic nature and their potential be loaded with a variety of drugs, including doxorubicin and paclitaxel along with various contrast agents, such as dyes and plasmonic nanoparticles. Antibodies targeting molecular markers of diseased cells can be conjugated to the surface of the liposomes to initiate receptor mediated endocytosis by diseased cells; this uptake can result in the release of the liposomal payload. In turn, the uptake of liposomes can result in release of the dyes encapsulated within the lipid shell or core (for example through the same mechanisms that have been actively explored for liposomal drug delivery inside cells) that will lead to profound changes in dye optical properties. These changes can be monitored by a variety of optical imaging techniques ranging from traditional optical microscopy to near infrared spectroscopy, multispectral imaging, photoacoustic imaging and fluorescence.

In general, unlike “traditional” contrast agents, polymethine dye-aggregate loaded liposomes may have the ability to interact with and sense cellular induced behavior (like endocytosis via receptor-mediated binding) via their changing optical absorption spectra and/or fluorescence. Furthermore, if polymethine dye-aggregate loaded liposomes, or liposomes loaded specifically with indocyanine green (ICG) J-aggregates, are also loaded with drugs, then the same change in optical properties of the dye that can be monitored to sense cellular behavior, can also confirm drug delivery at a cellular level, making polymethine dye-aggregate loaded liposomes a unique and ideal choice for molecular and cellular specific, sensitive imaging.

### SUMMARY

The present disclosure generally relates to cellular sensing and drug delivery. More specifically, the present disclosure relates to methods of detecting cellular function and/or targeted drug delivery in biological tissues, biological organisms in general and biological systems using dye-loaded liposomes.

An embodiment of the present invention provides a method for sensing dissociation of dye-aggregates due to the rupture of particles consisting of the dye in liposomes, based on changes in their absorption spectra. According to this embodiment, the method includes a step of monitoring the first and/or second peak absorbance ranges of about 870-920 nm (aggregate peak) and about 760-810 nm (monomer peak). An increase or decrease of either peak by more than at least 2 percent of the baseline values may be used to detect the extent of dissociation activity. In other embodiments, such extent may be determined by performing ratiometric analysis of the characteristic aggregate and monomer peaks to determine degree of liposomal rupture. The liposomal particle may comprise a liposome, a coating on the surface of the liposome, with or without a targeting moiety within the coating and a plurality of dye-aggregates within the liposome. Upon rupture of the liposome, the aggregates may be dissolved by the surrounding media or intracellular components, inducing a decrease in the aggregate peak with respect to that of the monomer.

Further methods of detecting the extent of dissociation activity include a step of monitoring absorbance spectra and detecting of reduction in absorbance peak in the first wavelength range (aggregate peak), such absorbance being at or below a first predefined threshold, such as for example about 2 percent, 3 percent, 4 percent, 5 percent, 6 percent, 7 percent, 8 percent, 9 percent, or 10 percent reduction or greater of the baseline level of the absorbance spectra.

In other embodiments, the extent of dissociation activity may be further detected by monitoring the increase in the second wavelength range corresponding to the monomer peak of the dye. In this case, dissociation of aggregates may be established after detecting an increase in absorbance peak in the second wavelength range at or above a second predefined threshold, such as for example about 2 percent, 3 percent, 4 percent, 5 percent, 6 percent, 7 percent, 8 percent, 9 percent, 10 percent increase or more over the baseline level of the absorbance spectra.

Another embodiment provides a method for sensing dissociation of dye-aggregates due to the rupture of particles consisting of dye in aggregate form encapsulated in liposomes, based on changes in their fluorescence, which can be monitored using near infrared fluorescence among other techniques. According to this embodiment, the method comprises a step of monitoring the fluorescence of the dye-loaded liposomal constructs to detect an increase or decrease in signal upon rupture. The liposomal particle may comprise a liposome, a coating on the surface of the liposome, with or without a targeting moiety within the coating, and a plurality of dye-aggregates within the liposome. Upon rupture of the liposome, the aggregates may be dissolved by the surrounding media or intracellular components, inducing an increase in fluorescence due to a reduction in self-quenching.

Yet another embodiment provides methods of detection of cellular uptake, with or without targeted delivery or a therapeutic or diagnostic agent. According to this embodiment, the method comprises a step of introducing targeted liposomes to a biological tissue or system to detect cellular uptake based on the dissociation of dye-aggregates after uptake and liposomal rupture. The targeted liposomal particle may in that case comprise a liposome, a coating on the surface of the liposome, a targeting moiety within the coating, a plurality of dye-aggregates within the liposome, and with or without a payload, such as a drug that is housed within or tethered to the lipids in the liposome itself. Upon cellular uptake of the particle, the liposome may rupture and the aggregates may be dissolved. The absorption spectra and/or fluorescence may be monitored via imaging techniques, such as photoacoustic imaging or fluorescence imaging or a variety of other optical imaging techniques, to detect the changes highlighted in the previously mentioned embodiments and confirm targeted uptake and payload delivery.

### DRAWINGS

Some specific example embodiments of the disclosure may be understood by referring, in part, to the following description and the accompanying drawings.

FIG. 1 is an illustration of an example embodiment of a J-aggregate loaded liposome and the sensing scheme of the present invention.

FIG. 2 depicts a typical absorption spectra of ICG J-aggregates encapsulated in liposomes and a typical spectra after liposomal rupture by a surfactant such as Triton X.

FIG. 3 shows an exemplary fluorescence spectra of free ICG and the ICG J-aggregate loaded liposomes, both intact and ruptured.

FIG. 4 contains fluorescence microscopy images of cells that have uptaken the liposomal constructs of the invention.

FIG. 5 shows the absorption spectra of the novel system throughout cellular incubation with macrophages.

FIG. 6 shows an example of a local spectral change without relying on Triton X.

FIG. 7 displays the typical photoacoustic response over 1000 laser pulses, illustrating the stability of the construct.

FIG. 8 charts the photoacoustic intensity vs laser fluence, normalized to the mass of absorber in the sample for selected particles. The inset shows the response from 0-5 mJ/cm².

FIG. 9 charts the photoacoustic response for intact liposomes containing ICG J-aggregates versus free ICG-aggregates.

FIG. 10 shows the ratiometric shift without relying on complete breakdown of liposomes.

FIG. 11 shows the photoacoustic spectral scans of phantoms of ICG J-aggregate encapsulated in liposomes mixed with macrophage cells, and

FIG. 12 shows the ultrasound and photoacoustic images of the phantoms at relevant wavelengths of 790 nm and 890 nm.

### DESCRIPTION

The present disclosure provides, according to certain embodiments, methods for sensing the dissociation of dye aggregates due to the rupture of particles consisting of said dye in liposomes. Such particles may be used as contrast agents and sensors to monitor cellular activity or drug delivery for various imaging techniques. Although the sensing methods are described below utilizing ICG J-aggregate loaded liposomes, the methods of the present disclosure may utilize other aggregate forming dyes of the polymethine class.

In accordance with embodiments of the present invention, provided is a dye-aggregate loaded liposome. Additionally, embodiments of the present invention provide methods of sensing by monitoring the absorption and/or fluorescence spectra of these particles via a variety of imaging techniques. Such imaging techniques may include ultrasound imaging, photoacoustic imaging, fluorescence imaging, near infrared fluorescence imaging, near infrared spectroscopy imaging, two-photon luminescence, optical coherence tomography imaging, and optical frequency domain imaging. In embodiments, such imaging may be conducted in vivo (for example using an intravascular catheter-based imaging probe, laparoscopic device, or a transcutaneous imaging probe) or using an ex-vivo assay. Such imaging may also be conducted during surgery for a purpose of spatial guidance, for a diagnostic purpose, or for a purpose of monitoring of disease recurrence. The imaging may be done using either a laparoscopic device, an endoscope, or a surface imaging probe.

One of the many advantages of the present invention is that the size of the liposome may be selected to allow for passive diffusion into tumor tissues, and therefore, may be easily used for therapy and imaging for many pathologies. The small size of the particle may allow the liposome to travel almost anywhere in the body where therapy and/or imaging may be required. For example, embodiments incorporating therapeutic agents could act as drug delivery and drug release systems for active cellular uptake sensing.

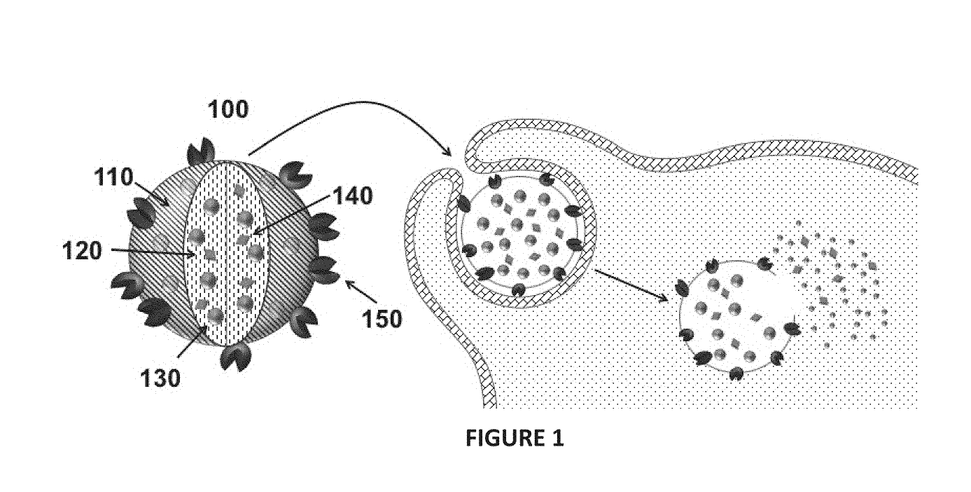

Another advantage of the current invention is the robustness of the sensing method. The liposomal constructs are highly stable due to the inclusion of aggregates. The large aggregates prevent dye leakage allowing for consistent delivery of the full payload to cells. The current invention relies upon the disruption of liposomes within cells or disruption due to cellular activity. The aggregates may then be dissolved due to a decrease in local concentration upon rupture. FIG. 1 illustrates this release of liposome payload upon cellular interaction. This consistent dissolution provides reliable spectral shifts and fluorescence changes to detect cellular uptake and confirm delivery. The amphiphilic nature of the dye allows strong interactions with the lipid shell, maintaining solubility and preventing the formation of insoluble forms of the aggregates.

An additional advantage of the current invention is utilizing a construct that is completely biocompatible and easily cleared after detection. By utilizing indocyanine green (ICG) dye and lipids, all components of the construct have been approved by the Food and Drug Administration for use within the human body for other applications. Numerous other dyes within the polymethine class are currently undergoing clinical trials with encouraging results. Upon rupture, the lipid shell is dispersed, likely incorporating into cell walls or getting repackaged by the cell, and the dye is cleared soon after by the renal system, giving a distinct advantage over other contrast agents that may accumulate in the liver and spleen. The last dissolution of dye upon rupture provides instant feedback on the extent of the cellular uptake and interaction.

Further advantages of the current invention include applicability to various cellular systems. The liposomes may be functionalized with antibodies, antibody fragments, folates, aptamers, vitamins, and/or polymers, allowing targeting of a wide range of cell types. As the sensing mechanism depends upon breakdown of the liposome and associated aggregates upon cellular uptake, this creates a possibility for targeting cells outside of biological tissue; particularly, harmful biological agents. Bacterial targeting could be employed coupled with the sensing mechanism to provide a rapid spectral detection method for released biological agents, acting as a warning system in bioterrorism or biological warfare.

Atherosclerosis and cancer are two examples of advantageous use of the therapeutic or diagnostic methods of the present invention. In atherosclerosis for example, antibodies of the invention may be used to target macrophages, foam cells, or other inflammatory cells in plaques, as well as other white blood cells, smooth muscle cells, or endothelial cells. Antibody targets may further include folate receptor beta, markers of apoptosis (annexins), markers of glucose uptake, proteinases (such as MMPs), multiple clusters of differentiation including CD36, and CD68. Other molecular targets of interest may include P-selectin, VCAM-1, ICAM-1, VLA-4, JAM-A, Connexin 43, CCL2(MCP-1)/CCR2, CCL5(RANTES)/CCR5, CX3CL1(fractal-kine)CX3CR1, and MIF. Further target applications in atherosclerosis may be found in the article by Meyer I D, Martinet W, and Meyer G entitled “Therapeutic strategies to deplete macrophages in atherosclerotic plaques”, British Journal of Clinical Pharmacology, 74;2:246-263, 2012, incorporated herein in its entirety by reference.

Various types of therapeutic agents may be used for the methods of the present invention. Examples of such agents include clodronate, lithium chloride, recombinant TRAIL, NO donor, thapsigargin and tunicamycin, cycloheximide and anisomycin, mTOR inhibitor (everolimus), imiquimod, glucocorticoids, CpG oligonucleotides, clot-dissolving drugs, and RNA in general.

In the field of oncology, the present invention may find uses with antibodies used to target cancer cells or inflammatory cells. A long list of suitable antibody targets may be considered to include those on cancer cells and inflammatory cells. Examples may include folate receptor beta, markers of apoptosis (annexins), markers of glucose uptake, proteinases (such as MMPs), multiple clusters of differentiation including CD36 and CD68, P-selectin, VCAM-1, ICAM-1, VLA-4, JAM-A, Connexin 43, CCL2(MCP-1)/CCR2, CCL5(RANTES)/CCR5, CX3CL1(fractal-kine)CX3CR1, MIF, EGFR, TGF beta, folate receptors in general. PSMA, HER2, VEGF, interleukin 4, interleukin 10, and interleukin 13.

Examples of suitable therapeutic agents for cancer treatments may include chemotherapeutic drugs in general such as paclitaxel, doxorubicin, cisplatin, CpG oligonucleotides, and RNA in general. Immunotherapies can also be delivered such as trastuzamab, lapatinib, etc. A more exhaustive list of suitable therapeutic agents is described by Dougan, M. & Dranoff, G. in “Innate Immune Regulation and Cancer Immunotherapy” (ed. Rongfu Wang) p. 391-414 (Springer N.Y., 2012), which is incorporated herein by reference in its entirety.

Sensing techniques of the current invention differ from prior technologies in a number of aspects. First, the method relies on a change in conformation of an aggregate forming dye. This conformational change is forced upon disruption of the liposomal particle, creating a consistent shift in absorption spectra and fluorescence. Second, the method utilizes a particle effective for either or all of imaging, sensing cellular function, and drug delivery. Current known technologies allow targeted delivery and imaging, but the present invention may also include built-in sensing to monitor cellular uptake.

A sketch of one embodiment of the invention is illustrated in FIG. 1. The sensing method requires a dye-aggregate loaded liposome, such as could be used for imaging or therapy purposes. A dye-aggregate loaded liposome 100 may comprise a central liquid 120, which in turn may comprise water or a buffer, such as phosphate buffered saline. The liposome 100 may further comprise an aggregated polymethine dye 130, which may in turn comprise indocyanine green (ICG). In embodiments, more than one polymethine dye may be present. The liposome 100 may further comprise at least one or more therapeutic agent 140, such as doxorubicin, cisplatin, paclitaxel or other approved therapeutic agents, including at least atherosclerosis and cancer treatments, for example as cited above. The liposome 100 may further comprise a lipid shell 110. which may comprise one or more lipids, such as DPPC, DSPE-PEG, DSPE-PEG-Mal and cholesterol. Other suitable lipids may include natural lipids, sphingolipids, phospholipids, sterols, bioactive lipids, coenzyme A & derivatives, fatty acid modified lipids, headgroup modified lipids, fluorescent lipids, polymerizable lipids, cationic lipids, and neutral lipids.

A suitable therapeutic agent may be incorporated into a liposome in a number of ways, for example it may be contained within the core of the liposome, in the lipid bilayer, tethered to the lipid, or adhered either through covalent or electrostatic binding to the coating or lipid.

The liposome 100 may further comprise a coating 110, which may for example comprise bovine serum albumin (BSA), Polyethylene glycol (PEG), carbohydrates, or dextran, or combinations thereof. The coating 110 may further comprise one or more targeting mechanisms 150, such as antibodies, antibody fragments, folates, aptamers, vitamins, and/or polymers.

The sensing method of the invention may comprise detection of absorbance, which may utilize one or more imaging techniques mentioned above, such as photoacoustic imaging or UV-Vis-NIR (ultraviolet to visible to near infrared) spectroscopy. The sensing method may also comprise detection of fluorescence, which may utilize a suitable imaging technique, such as two-photon fluorescence, near infrared imaging, or fluorescence microscopy. The sensing method may further comprise visual detection, which may utilize an optical instrument such as optical microscopy or optical coherence tomography.

For the purposes of this description, a peak in absorbance spectra may be defined as a local maximum of absorbance intensity in the absorption spectra, which may be identified by a value of zero in the first or second derivative of the spectra. This may incorporate one or both local maxima and changes in concavity of the absorption spectra. Signal of interest may be at least 15% greater than noise in order to assure accurate observation and identification of the peak. In embodiments, an increase or a decrease of the baseline level by at least 2 percent may be considered as defining a local peak or valley. In other embodiments, a threshold to define a peak may be established as 5 percent of the baseline level change and in further embodiments that threshold may be established at 5 to 10 percent or more of the baseline level.

As noted above, the biocompatible dye may be any dye that forms aggregates resulting in a shift in their absorption spectra or fluorescence. Examples of suitable dyes that form these aggregates may include polymethines, including cyanines such as ICG, squaraines, and perylene bismides. Various polymethine dyes may be useful for the purposes of the present invention. Exemplary classes of polymethine dyes that are known to form either or both J and H aggregates at various concentrations are cyanines, merocyanines, squaraines, and rylenes. As concentration of the dye in solution increases, the dyes progress through H-aggregate polymeric formation yielding a hypsochromic (blue) shift in absorption spectra with a broad absorption peak. The H-aggregates may be composed of plane-to-plane organization of the dyes at a molecular level. When dye concentration is further increased, molecules organize head-to-tail, therefore forming J-aggregates. This supramolecular organization induces a bathochromic (red) shift in absorption with a sharp absorption peak.

Examples of cyanine dyes useful for the purposes of the invention may include an indocyanine green, Cy3, Cy3.5, Cy5.5, and Cy7. Useful examples of merocyanine dyes may include a pseudoisocyanine chloride and merocyanine I. Useful examples of squaraine dyes may include a squarylium dye III. Useful example of rylene dyes may include a perylene bismide.

The terms “coat,” “coated,” or “coating,” as used herein, also refer to at least a partial coating of a particle. One hundred percent coverage of a particle is not implied by these terms. Rather, a droplet may be coated if it has at least a partial coating.

To facilitate a better understanding of the present invention, the following examples of certain aspects of some embodiments are given. In no way should the following examples be read to limit, or define, the scope of the invention.

### EXAMPLE 1

### Dissolution of J-Aggregates upon Liposome Disruption

Liposomes of the invention may be synthesized via methods similar to those presented previously in literature. Specifically, the example outlined here utilized indocyanine green (ICG) J-aggregates loaded in liposomes to form 100 nm particles. Samples were purified to remove free ICG before further studies. Particles were stable after synthesis and stored in PBS, and monitored via ultraviolet-to visible-to near infrared (UV-Vis-NIR) light absorption spectrophotometry and fluorometry; these two techniques will be discussed here.

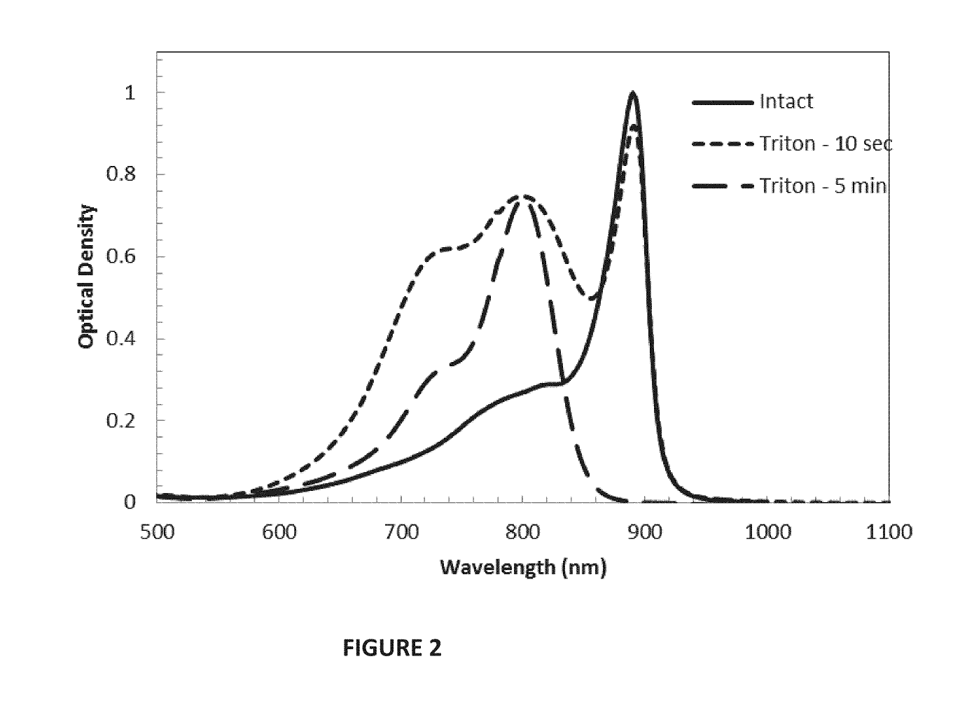

UV-Vis-NIR spectrophotometry was performed on a spectrophotometer with a 5 nm slit width, scanned over the wavelengths of 400-1100 nm, sampled every 1 nm, at 3 nm per second. Samples were prepared by diluting the ICG-loaded liposomes with water to a 0.1 mg/mL lipid concentration and loading 2.5 mL in a plastic cuvette. To disrupt the liposomes, a common nonionic liquid surfactant, Triton X-100, was used. A 10 vol % solution of Triton X-100 in water was prepared and added to the liposome solution in a 1:1 ratio. The UV-Vis-NIR spectra were collected before, during, and after disruption. As seen in FIG. 2, the UV-Vis-NIR spectra shows that as-made liposomes with encapsulated J-aggregates of ICG display absorption peak at about 900 nm. Generally speaking, in the intact sample, a sharp peak is present at about 891 nm. This peak is very narrow due to the formation of J-aggregates within the liposomes, creating a tight size and structure distribution. Upon addition of the Triton, spectral changes occur quickly. Immediately, the 891 nm peak begins to decrease in strength as the 790 nm peak increases due to the disruption of liposomes and the breakdown of J-aggregates. After 5 min, all the liposomes were completely destroyed and the ICG was present in its free monomer form.

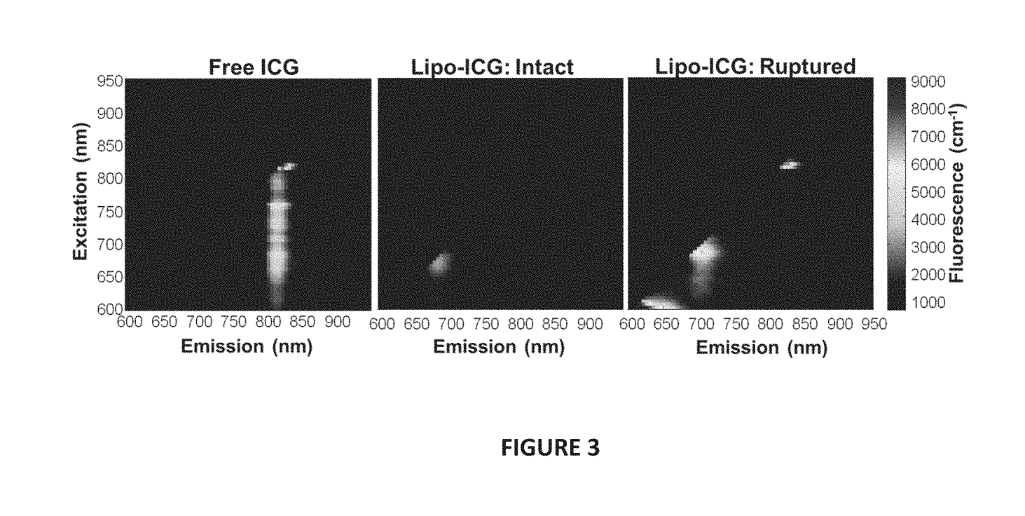

The fluorescence spectra was also collected for the constructs. The fluorescent intensity was calibrated with respect to the 397 nm (3380 cm⁻¹) Raman peak of water. Samples were prepared with a constant ICG concentration of 0.025 mg/mL. ICG-loaded liposomes with water to a 0.125 mg/mL lipid concentration, yielding a 0.025 mg/mL total ICG concentration. To disrupt the liposomes, a common nonionic liquid surfactant, Triton X-100, was used. A 10 vol % solution of Triton X-100 in water was prepared and added to the liposome solution in a 1:10 ratio, 10 min before measurement. The fluorescent spectra were collected for free ICG and before and after disruption of the ICG J-aggregate loaded liposomes. As seen in FIG. 3, the fluorescent spectra shows free ICG emits from 810-825 nm after stimulation by light at 830 nm or lower. The as-made liposomes with encapsulated J-aggregates of ICG exhibit low emission from 670-690 nm after 690 nm stimulation. After disruption, there is a sharp increase in fluorescence from 810-825 nm, corresponding with the free ICG spectra. Likewise, there is another fluorescent peak at 690-710 nm. This rapid increase in fluorescence indicates the breakdown and release of J-aggregates upon liposome disruption, resulting in a fluorescence spectra similar to that of free ICG.

This example further illustrates an opportunity for a general, non-biologic use of the methods of the invention to detect a surfactant (like Triton X) in a liquid in the presence of liposomes with encapsulated J-aggregates of ICG.

### EXAMPLE 2

### Disruption of Liposomes by Cellular Uptake

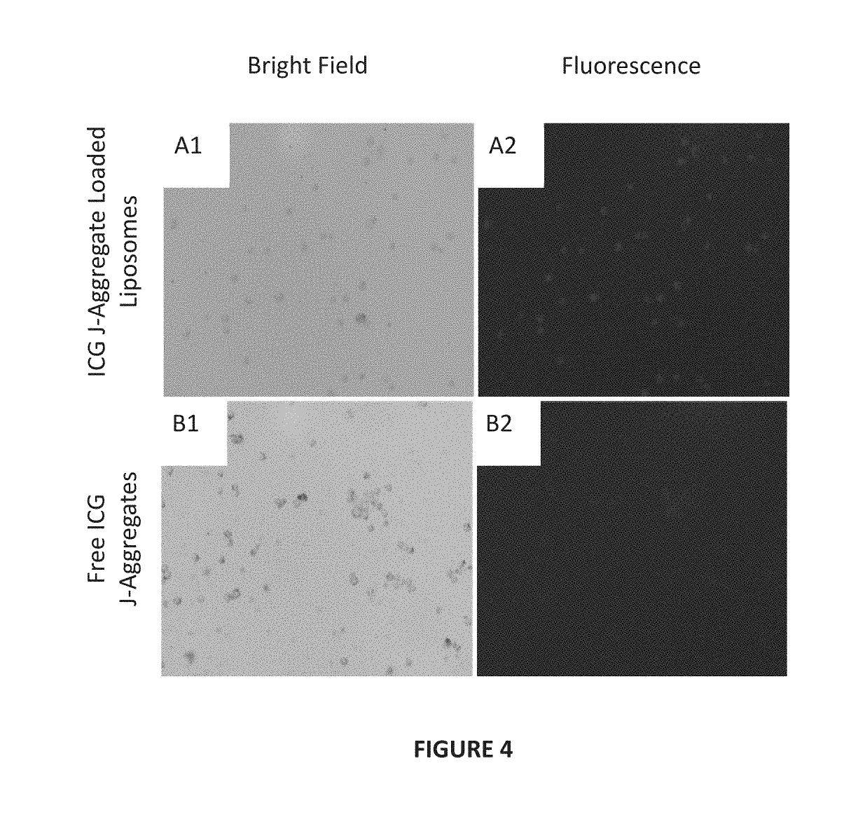

ICG J-aggregate loaded liposomes were tested for their applicability as cellular uptake sensors. About 50,000 J774 A.1 macrophages (ATCC) were seeded onto coverslips in a 6-well plate. ICG J-aggregate loaded liposomes and free ICG J-aggregates were added to the cell media for 2 hours to allow uptake by cells. Following uptake, coverslips were washed with PBS to remove any free dye or liposome, and then imaged using a 75 W Xenon light source and Leica DM6000 upright microscope. Fluorescence images were acquired with a 600 nm excitation and a red low pass filter. Images were collected through a 20×0.5NA objective and detected using a SPOT 1.4MP color mosaic camera. FIG. 4 shows the microscopy images acquired during this study. A1 and A2 show the bright field and fluorescence images, respectively, for cells incubated with ICG J-aggregate loaded liposomes. As seen, there is significant signal correlating with the cells in the fluorescence image. Likewise, B1 and B2 show the bright field and fluorescence images, respectively, for cells incubated with free ICG J-aggregates. Though there is significant background fluorescence, there is very little fluorescence correlating to the cells. The lack of fluorescence in the cells incubated with free ICG is due to the presence of stable J-aggregates that are not easily broken by the cells. However, cells incubated with ICG J-aggregate loaded liposomes display fluorescence due to the disruption of the liposomes after cellular uptake, followed by the release and dissolution of the J-aggregates, as observed in the previous Triton X-100 experiments.

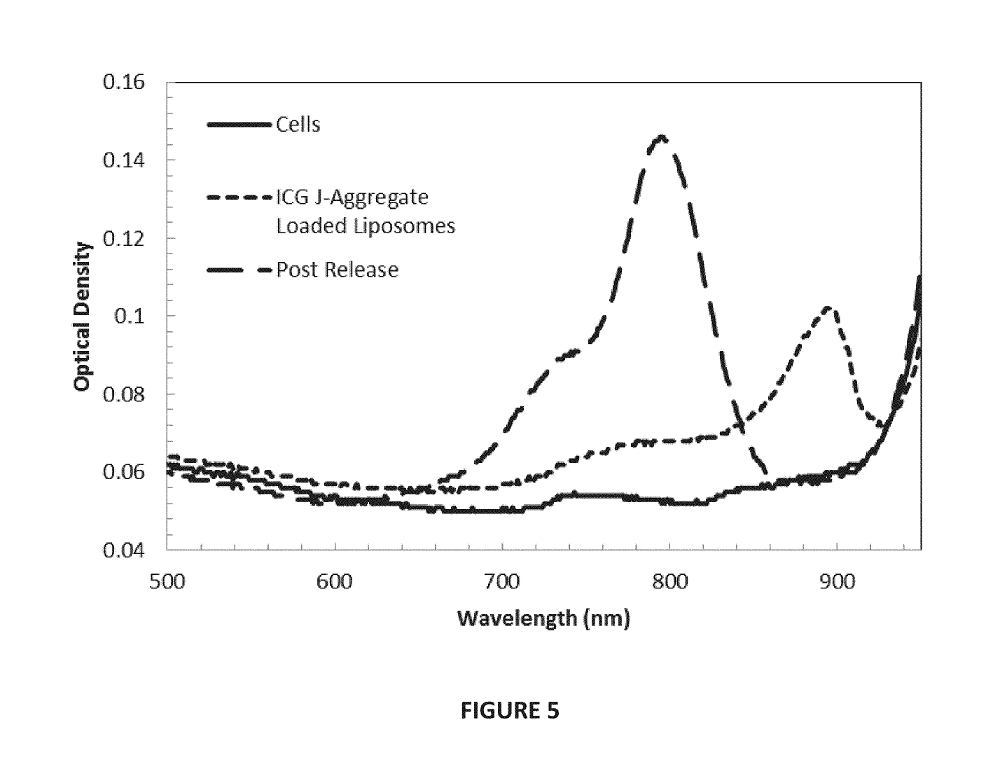

For spectral analysis, J774 A.1 cells were plated on 96 well plates at 10⁵ cells per well. 100 μL of phenol free media was added to each well and the cells were incubated 24 hrs at 37° C. in 5% CO₂. The media was aspirated, and either ICG J-aggregate loaded liposomes, fresh ICG, or free ICG J-aggregates were added to the cells in phenol free growth media. Cells were then incubated for 4 hrs. After incubation, the cells were rinsed 6 times with phenol free media. The UV-Vis-NIR spectra was collected using a BioTek plate reader. The results can be seen in FIG. 5. After 4 hrs, the cells had an absorbance at 890 nm indicating effective uptake of our liposomes. After rupture with TritonX, the 890 nm peak was observed to disappear and the 780 nm peak was observed to arise. Three different liposome-to-cell ratios were also tested, but the final absorbance spectra for all three were the same. This indicates that the final liposome uptake by the cells was limited by rate of cell cellular uptake and was not concentration-dependent at the levels tested. Overall, the ICG J-aggregate loaded liposomes were uptaken by macrophages efficiently.

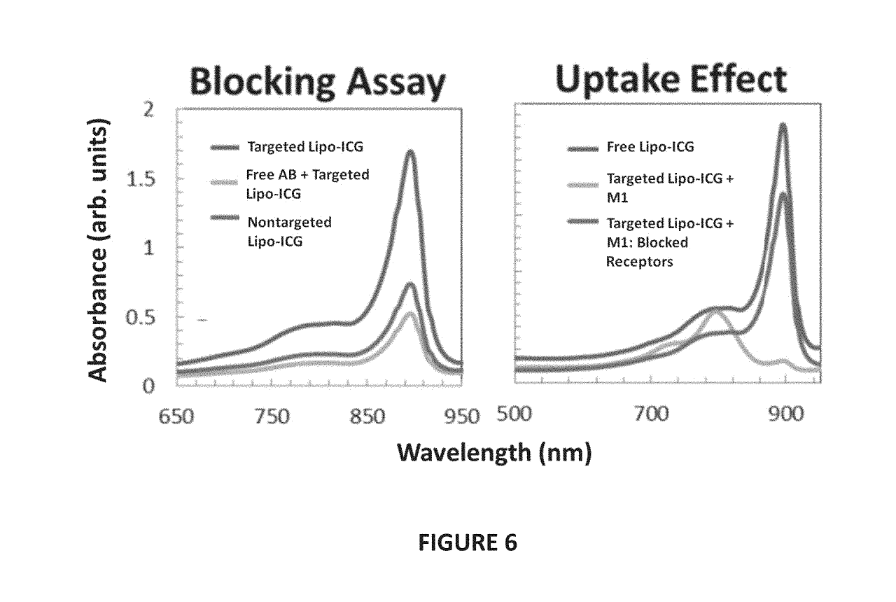

In embodiments, reliance on Triton may not be necessary. FIG. 6 shows an example of absorption spectra of M1 macrophages after 24 hr incubation with (a) FRB targeted ICG J-aggregate loaded liposomes, (b) free a-FRB followed by FRB targeted ICG J-aggregate loaded liposomes, and (c) non-targeted ICG J-aggregate loaded liposomes. (D) Absorption spectra of (d) ICG J-aggregate loaded liposomes, (e) supernatant after incubating the targeted particles with M1 activated macrophages, and (f) supernatant after incubating targeted particles with FRB blocked M1 activated macrophages. The blocking assay was performed to determine the effect of our a-FRβ on targeting M1 activated macrophages. These were performed by feeding 10 ng/mL concentration of a-FRβ in media to a flask of macrophages and letting it incubate for 24 hrs. At this point, the FRβ sites were effectively blocked, thereby inhibiting binding between our targeted construct and the cells. The ICG-loaded liposome constructs were then added to the flask and allowed to incubate for 24 hrs. Cells were scraped from the flasks and concentrated to 3×10⁶ cells/mL and fixed with formalin for UV-Vis studies. Supernatants were also analyzed. FIG. 6 shows a sample of results from these studies. As seen, by blocking the FRβ sites we negate our targeting and ICG-loaded liposomes were uptaken at approximately the same rate for both targeted and non-targeted particles. UV-Vis shows that the absorption peak remained at 890 nm, indicative of J-aggregates. After checking the spectra of the supernatant it was clear that our J-aggregates were broken down during the 24 hr incubation time, but only by the cells with available FRβ binding sites; the blocked macrophages showed no spectral shift in the supernatant. This indicates that the breakdown of the aggregates is dependent upon the uptake pathway. Upon breakdown, the ICG was ejected into the media, no longer in J-aggregate form (780 nm peak). Based on the small 890 nm peak remaining in the media, we were able to quantify that 90% of the ICG-loaded liposomes were uptaken by the M1 activated macrophages, with only 20% uptaken in the blocked case.

### EXAMPLE 3

### Photoacoustic Imaging of Liposomes

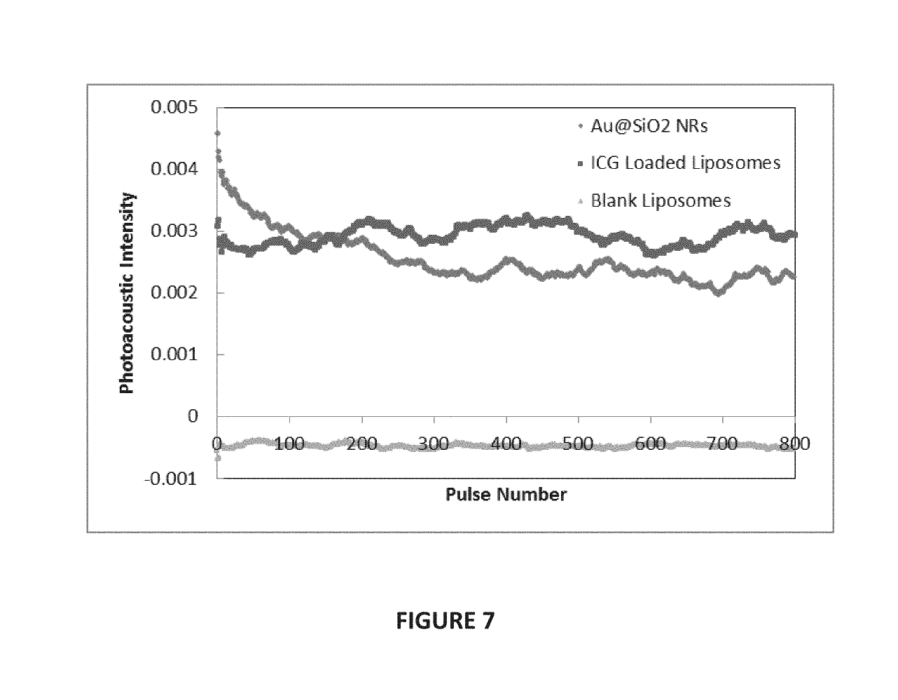

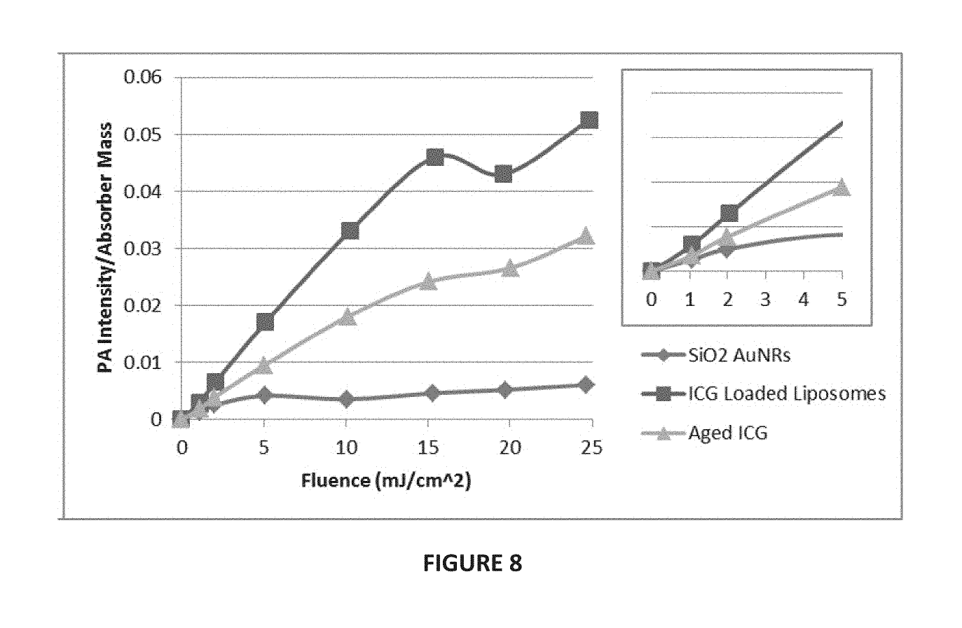

The ICG J-aggregate loaded liposomes may be effectively utilized as a photoacoustic contrast agent. Photoacoustic (PA) imaging was performed to determine the PA response and stability of the ICG J-aggregate loaded liposomes. Each sample was exposed to seven different fluences to determine the stability of the nanoparticles over 900 laser pulses at each fluence. Each sample was first subjected to 900 pulses at 1 mJ/cm². Then the same sample was subjected to 900 pulses at 2 mJ/cm². This was repeated for 5, 10, 15, 20, and 25 mJ/cm². Characteristic PA response curves can be seen in FIG. 7 for a laser fluence of 5 mJ/cm². Samples were compared to silica-coated gold nanorods as a standard. As may be expected, the silica-coated gold nanorods have a higher initial response; however, liposomal constructs of the present invention produce a very stable signal while the nanorods have a sharp initial signal dropoff. Any decrease in the PA signal with subsequent pulses indicates that irreversible changes to the optical absorption of the construct was occurring. The liposomal constructs are suitable for in vivo imaging since their signal strength is high and is maintained over many pulses. To illustrate the stability, the average of the last 100 pulses at each fluence was plotted normalized to the mass of absorber (FIG. 8). A deviation in the curve from linear indicates the fluence at which the nanoparticle undergoes irreversible damage. These curves show that ICG loaded liposomes of the present invention exhibit high stability, with a nearly linear plot. The normalized data indicates that these ICG-loaded liposomes exhibit enhanced performance over free J-aggregates, and due to their stability, also outperform silica-coated gold nanorods.

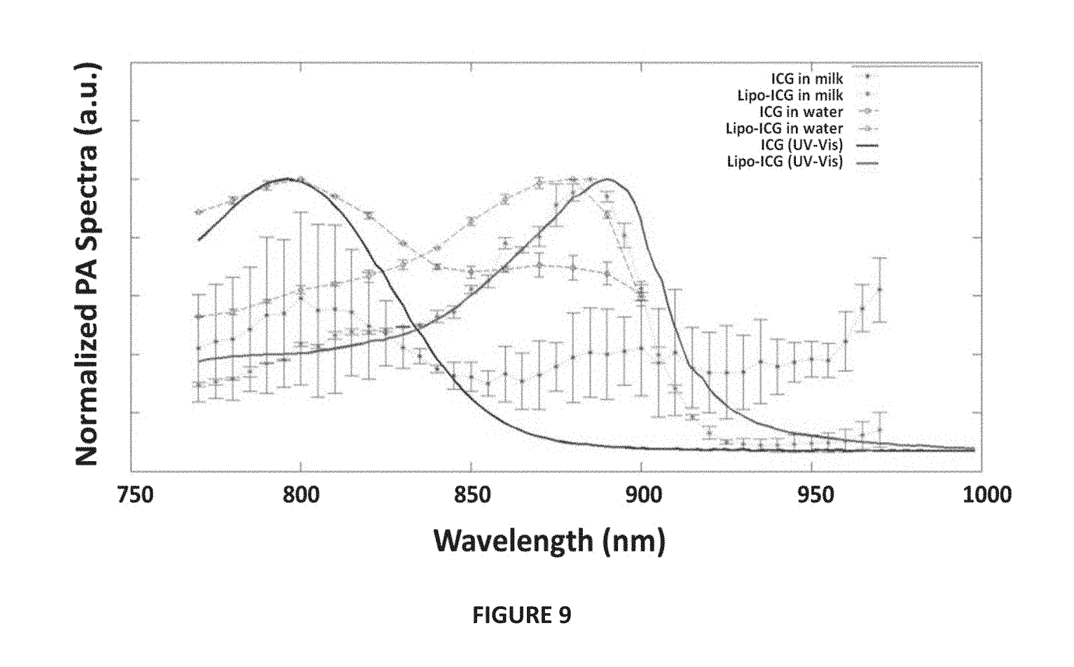

The liposomes were imaged both in water and milk (whole, 3.25% fat) phantom to demonstrate feasibility in fatty environments. Samples were deposited in 3 mm diameter tubing and submerged in either water or milk. All images and spectra were acquired on a Vevo LAZR (VisulSonics, Toronto, Canada) system using a 21 MHz pre-clinical transducer with a 2.4 cm field of view, sub-millimeter spatial resolution, and a 5 Hz frame rate. For initial spectral characterization in water, images were acquired at wavelengths from 650-970 nm with a gain of 37 dB and persistence of 8. In milk, the gain was optimized to reduce background signal and set to 35 dB. Images were acquired at wavelengths of 650-970 nm with persistence of 8. As seen in FIG. 9, the peak absorption was observed at approximately 800 nm for free ICG and 875 nm for ICG J-aggregate loaded liposomes.

### EXAMPLE 4

### Photoacoustic Imaging of Cell Phantoms with ICG J-Aggregate Loaded Liposomes

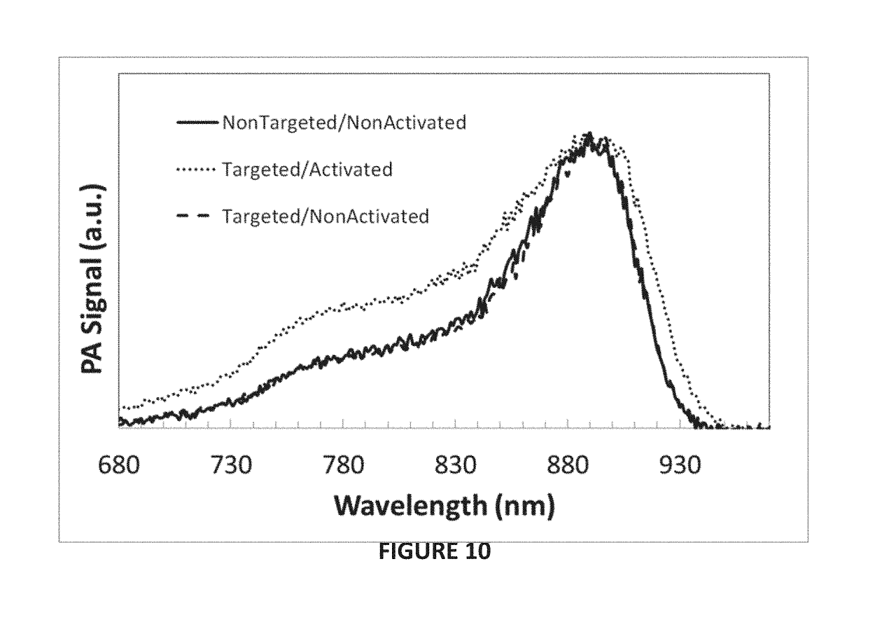

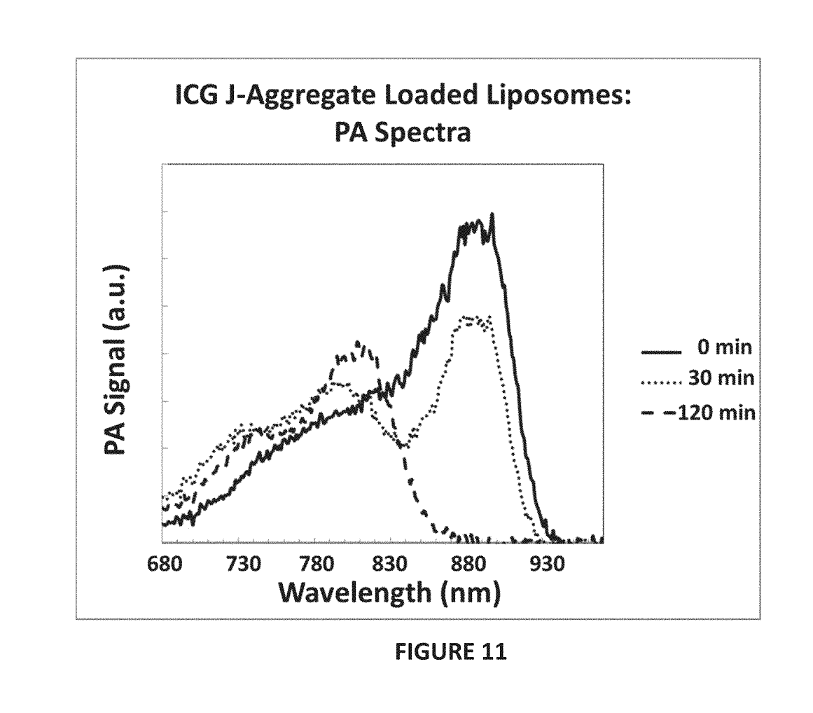

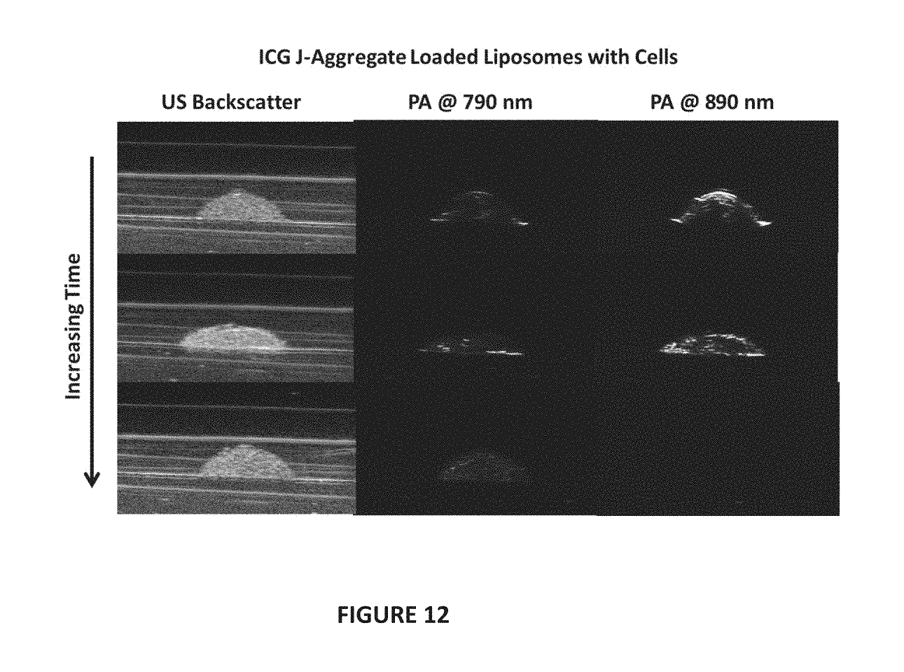

Cell phantoms were constructed to demonstrate the capabilities of the ICG J-aggregate loaded liposomes as contrast agents and sensors in a biological environment. Phantoms were constructed as follows. A 4% gelatin base was formed by dissolving 4 g of gelatin per 100 mL of water at 60° C. Macrophage cells (J774 A.1, 10⁷/mL) were mixed with the ICG J-aggregate loaded liposomes, and with an absorbance of 10 OD at 890 nm for a 1 cm pathlength, in a 3:1 ratio. For some samples, a 100 μL cell/liposome solution was mixed with 5 μL of Triton X-100 to mimic activated macrophages and stimulate liposome disruption and incubated for 2 hours. An 8% gelatin solution was then mixed in a 1:1 ratio with the cell/liposome solution, and a 20 μL was placed on the set gelatin phantom base. Photoacoustic imaging was performed using Vevo 2100 and VevoLAZR, with a spectroscopic scan from 680-970 nm at a 1 mJ/cm² laser fluence. Spectral curves were generated by selecting a small region inside of the inclusion and averaging the intensity for every wavelength. FIG. 10 depicts the photoacoustic spectra of cells incubated with targeted and non-targeted ICG J-aggregate loaded liposomes and the associated ratiometric spectral shift upon uptake of targeted liposomes. FIG. 11 shows the results of the spectral scans before and after disruption with TritonX. FIG. 12 shows the ultrasound and photoacoustic images of the phantoms. The data shown have been background corrected versus a cell-only phantom. As seen, the ICG J-aggregate loaded liposomes perform as expected, with their photoacoustic spectra mapping to their absorbance spectra. After incubation with Triton X-100 in the presence of macrophage cells, the 890 nm peak decreases and 790 and 700 nm peaks appear. After 2 hrs, the 890 nm peak disappears completely, indicating the complete dissolution of all J-aggregates. The photoacoustic images corroborate these findings, with the signal intensity decreasing at 890 nm with longer incubation times and increasing at 790 nm. These spectral shifts indicate utility of the dye-aggregate loaded liposomes as cellular sensors.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

What is claimed is:

1. A method for detecting dissociation of an aggregate form of a polymethine dye into a monomeric form comprising a step of (i) detecting either a hypsochromic shift or a bathochromic shift in a peak wavelength of the absorbance spectra of said polymethine dye and/or (ii) detecting a change of fluorescence signal above a predetermined fluorescence change threshold, said fluorescence signal is generated by said polymethine dye in said monomeric form.

2. The method as in claim 1, wherein said steps (i) or (ii) are conducted using one or more of the types of imaging selected from a group consisting of ultrasound imaging, photoacoustic imaging, fluorescence imaging, near infrared fluorescence imaging, near infrared spectroscopy imaging, two-photon luminescence, optical coherence tomography imaging, and optical frequency domain imaging.

3. The method as in claim 2, wherein said imaging in steps (i) or (ii) is conducted in-vivo or using an ex-vivo assay.

4. The method as in claim 2, wherein said imaging is conducted intravascularly using a catheter imaging probe.

5. The method as in claim 2, wherein said imaging is conducted during surgery for a purpose of spatial guidance, for a diagnostic purpose, or for a purpose of monitoring of disease recurrence, said imaging is done using either a laparoscopic device, an endoscope, or a surface imaging probe.

6. The method as in claim 1, wherein said aggregate form of a polymethine dye is J-aggregates of indocyanine green, and said step of detecting includes a step of monitoring for appearance of absorbance spectra with a peak wavelength in a range from about 760 nm to about 810 nm.

7. The method as in claim 1, wherein said polymethine dye is selected from a group of dyes consisting of a cyanine dye, a merocyanine dye, a squaraine dye, and a perylene bismide dye.

8. The method as in claim 7, wherein said polymethine dye is provided in a form of a J-aggregate or an H-aggregate.

9. The method as in claim 7, where said cyanine dye is selected from a group consisting of an indocyanine green dye, Cy3 dye, Cy3.5 dye, Cy5.5 dye, and Cy7 dye.

10. The method as in claim 7, wherein said merocyanine dye is a merocyanine I dye.

11. The method as in claim 7, wherein said squaraine dye is a squarylium dye III.

12. The method as in claim 2, wherein said dissociation is detected once said absorbance spectra exceeds about 2 percent of a baseline level in a range from about 760 nm to about 810 nm.

13. The method as in claim 1, wherein said aggregate form of a polymethine dye is J-aggregates of indocyanine green, and said step of detecting includes a step of monitoring for a reduction by a predetermined margin in absorbance spectra peak wavelength in a wavelength range from about 870 nm to about 920 nm.

14. The method as in claim 13, wherein said detecting is established once said reduction in absorbance spectra exceeds about 2 percent of a baseline level of said absorbance spectra.

15. A method of detecting an extent of dissociation activity of an aggregate form of a polymethine dye, said method comprising a step of monitoring absorbance in a first wavelength range or a second wavelength range, said first wavelength range is defined by a predetermined peak absorbance range of intact sensor particles containing said polymethine dye in a form of an aggregate, said second wavelength range is defined by a predetermined peak absorbance range of ruptured particles when said polymethine dye aggregates are dissociated.

16. The method as in claim 15 further comprising the following steps:
a. providing a plurality of said sensor particles, each sensor particle encapsulating a polymethine dye provided in an aggregate form, said sensor particle when intact is characterized by said predetermined peak range in absorbance spectra, said sensor particle is further characterized by a spectral shift in absorbance spectra or a change in fluorescence above a predetermined fluorescence change threshold upon dissociation of said aggregates, and
b. monitoring for a presence of said spectral shift or said change in fluorescence as an indicator of rupture of said sensor particles and dissociation of said polymethine dye aggregates.

17. The method as in claim 15, wherein said step of monitoring absorbance includes detection of reduction in absorbance peak in said first wavelength range below a first predefined threshold.

18. The method as in claim 17, wherein said first predetermined threshold is about 2 percent reduction of the baseline level of said absorbance spectra.

19. The method as in claim 15, wherein said step of monitoring absorbance includes detection of an increase in absorbance peak in said second wavelength range above a second predefined threshold.

20. The method as in claim 19, wherein said second predetermined threshold is about 2 percent increase over the baseline level of said absorbance spectra.

21. The method as in claim 15, wherein said step of monitoring absorbance includes detection of a change in a ratio of absorbance peak in said first wavelength range to an absorbance peak in said second wavelength range by more than about 10 percent.

22. The method as in claim 15, wherein said aggregate form of the polymethine dye is J-aggregates of indocyanine green, said first wavelength range is from about 760 nm to about 810 nm, and said second wavelength range is from about 870 nm to about 920 nm.

23. The method as in claim 15 further including a step of detecting a harmful biological agent.

24. A method of detecting delivery of a therapeutic or diagnostic agent incorporated into a liposome, said method comprising the following steps:
a. providing a plurality of particles encapsulating said therapeutic or diagnostic agent and an aggregate form of a polymethine dye,
b. detecting dissociation of said polymethine dye aggregates by monitoring for (i) appearance of absorbance spectra with a peak wavelength in a range from about 760 nm to about 810 nm and/or (ii) reduction by a predetermined margin in absorbance spectra peak wavelength in a wavelength range from about 870 nm to about 920 nm,
wherein dissociation of said polymethine dye aggregates indicates rupture of said particles as well as release and/or delivery of said therapeutic agent in vivo.

25. The method as in claim 24, wherein said therapeutic or diagnostic agent is incorporated into a liposome by way of being contained within a core thereof, or in a lipid bilayer thereof, or tethered to said lipid, or adhered thereto either through a covalent binding or an electrostatic binding, said covalent binding or said electrostatic binding is formed to the coating thereof or said lipid.
